(19) United States
(12) Patent Application Publication
(10) Pub. No.: US 2016/0367710 A1
(43) Pub. Date: Dec. 22, 2016

(54) CONDITIONING SYSTEM FOR A STERILIZATION DEVICE, A STERILIZATION MACHINE AND A METHOD OF CONDITIONING A STERILIZATION DEVICE

(71) Applicant: TETRA LAVAL HOLDINGS & FINANCE S.A., Pully (CH)

(72) Inventor: Håkan MELLBIN, Hörby (SE)

(73) Assignee: TETRA LAVAL HOLDINGS & FINANCE S.A., Pully (CH)

(21) Appl. No.: 15/121,259

(22) PCT Filed: Jan. 21, 2015
(86) PCT No.: PCT/EP2015/051073

§ 371 Date: Aug. 24, 2016

(30) Foreign Application Priority Data
Feb. 25, 2014 (SE) 1450217-3

(51) Int. Cl. A61L 2/08; B65B 55/08

(52) U.S. Cl. CPC A61L 2/087; B65B 55/08; A61L 2202/23

(57) ABSTRACT

Conditioning system for a sterilization device, comprising a cooling system, at least one gas flow and a heat exchange unit, wherein the at least one gas flow is adapted to adjust the temperature of ambient gas around the sterilization device, wherein the cooling system comprises at least one medium flow, and wherein the at least one medium flow is adapted to cool and/or heat the at least one sterilization device, wherein the heat exchange unit is adapted to provide a heat exchange between the at least one medium flow and the at least one gas flow.

This invention relates to a conditioning system for a sterilization device, to a sterilization machine, in particular for packaging material and to a method of conditioning a sterilization device.

Electron beam irradiation has been considered as a promising alternative for sterilizing purposes. Electron beam irradiation provides sterilization of e.g. packaging material within e.g. a packaging machine. In general, an electron beam emitter is used that comprises an electron generator for emitting charge carriers, such as electrons. The electron generator comprises a cathode housing and a filament. When an electrical current is set through the filament, an electrical resistance of the filament causes the filament to be heated which causes the filament further on to emit a cloud of electrons. The electrons leave a housing of the electron beam emitter via an electron exit window. During the sterilization process, this electron exit window get heated. The prior art discloses cooling devices that are adapted to cool the electron exit window. However, cooling the electron exit window involves the risk that water vapour of the ambient air condensates at the e.g. cooled electron exit window. This is especially the case if the dew point of the ambient air is higher than the temperature of the electron exit window. The condensed water vapour can damage the sensitive electron exit window and in addition influence the sterilization properties. Furthermore, water drops could be formed which may drop on the material that is to be sterilized.

Therefore, it is an object of the current invention to provide a conditioning system, in particular for a sterilization device, a sterilization machine, in particular for packaging material and a method to condition a device, in particular a sterilization device, which avoids the drawbacks of prior art and which maintains an adaption of the ambient temperature surrounding the device and the temperature of the device itself, in particular to avoid condensation at an outer surfaces of the device.

This object is achieved by a conditioning system according to claim 1, by a sterilization machine according to claim 11 and by a method to condition ambient conditions of a device according to claim 12. Additional advantages and features of embodiments of the current invention are defined in the dependent claims.

According to the invention a conditioning system, especially for a sterilization device, comprises a cooling system, at least one gas flow and a heat exchange unit, wherein the at least one gas flow is adapted to adjust the temperature of the ambient gas surrounding the at least one device, in particular a sterilization device, wherein the cooling system comprises at least one medium flow, and wherein the at least one medium flow is adapted to cool and/or heat the at least one device, characterized in that the heat exchange unit is adapted to provide a heat exchange between the at least one medium flow and the at least one gas flow.

Expediently, the at least one gas flow is an air flow. Thus, the at least one gas flow is according to one or more embodiments gaseous. The medium of the at least one medium flow is expediently a liquid medium, such as water, or a specific coolant solution. However, the use of a gaseous medium is also possible. Generally, the elected medium for the at least one medium flow will depend on the heat transfer that has to be provided or realized by the medium. In the majority of cases a liquid (cooling) medium has a higher heat capacity. However, as already mentioned, it is also possible to use a gaseous medium if possible or necessary. The gas flow is adapted to adjust ambient conditions of the at least one device. In other words, the at least one gas flow is adapted to condition or control ambient conditions of the at least one device. Such ambient conditions can for example be the temperature of the ambient gas surrounding the device. If the pressure is constant, the humidity will be a function of the temperature. Ambient conditions are conditions surrounding the device.

The medium flow is adapted to cool the at least one device which means that e.g. heat that is produced by the at least one device can be absorbed and transferred by the at least one medium flow. However, cooling the device involves the risk that the ambient air around the device condensates at the device or on its outer surface, respectively, if the air around the device has a dew point that is higher than a temperature of the device or its outer surface. The dew point is the temperature at which the water vapour in air at constant barometric pressure condenses into liquid water at the same rate at which it evaporates. At temperatures below the dew point, water will leave the air. Thus, it is a big advantage that the heat exchange is realized between the at least one gas flow and the at least one medium flow, using the heat exchange unit. This allows to adjust or control the ambient conditions (around at least one device) in due consideration of the properties of the at least one medium flow that is used to cool the at least one device and vice versa. In other words, temperature levels of the at least one medium flow as well as of the at least one gas flow can be adapted so that the above mentioned problems, in particular concerning condensation, cannot occur.

In particular, the at least one medium flow controls or adjusts a temperature of the at least one gas flow via the heat exchange unit and vice versa. However, as the at least one medium flow is also used to cool the at least one device, a temperature of the at least one device or its outer surface, respectively, is always higher than a dew point/temperature of the ambient air of the device. More precisely, the device is cooled with a medium that has a temperature that is equal or higher than the ambient temperature of the device, i.e. equal or higher than the temperature of the ambient gas surrounding the device. In a first step, this is realized by the heat exchange unit which makes it possible to match, equalize or align the temperature of the at least one gas flow and the temperature of the at least one medium flow to each other. In other words, the heat exchange adapts or approximates the temperatures of the at least one gas flow and the at least one medium flow. However, according to one aspect of the invention, the at least one medium flow is then used to cool the at least one device which ensures that an outer surface of the device cannot be colder than its ambient temperature, i.e. than the gas surrounding the device. It this context, the advantage is that the at least one gas flow is also dried during the heat exchange which minimizes the risk of condensation even more.

The at least one device is a sterilization device comprising a power supply unit and at least one electron beam emitter that are connected to the power supply unit. Thus, it is also possible to connect more than one electron beam emitter to one power supply unit. The electron beam emitter comprises an electron generator for emitting charge carriers, such as electrons, along a path. The electron generator is generally enclosed in a hermetically sealed vacuum chamber. The vacuum chamber is provided according to one or more embodiments with an electron exit window. Furthermore, the electron generator comprises a cathode housing and a filament. In use, an electron beam is generated by heating the filament. When an electrical current is set through the filament, the electrical resistance of the filament causes the filament to be heated to a temperature in the order of 2.000° C.

This heating causes the filament to emit a cloud of electrons. The electrons are accelerated towards the electron exit window by means of a high voltage potential between the cathode housing and the electron exit window. Subsequently, the electrons pass through the electron exit window and continue towards a target area, e.g. a part of the packaging material that has to be sterilized. The high voltage potential is created by connecting the cathode housing and the filament to the power supply unit and by connecting the vacuum chamber to ground. The voltage that is supplied by the power supply unit lies, according to one or more embodiments, within a range of about 80 to 150 kV. However, higher and lower values are also possible.

An electron beam emitter as described before can be used for sterilization of packaging material or packages for food or drugs, biological or medical devices and so on. There are no limitations concerning the content of the packaging material. Thus, the content can be liquid, semi-liquid or solid. There are also no limitations concerning the use of the sterilization device of the electron beam emitter itself, respectively. Thus, the electron beam emitter or the sterilization device, respectively, can be used for inside and/or outside sterilization of e.g. packaging material, such as packaging containers e.g. for food, liquids or drugs. It goes without saying that it is very important to keep the outer surface of the sterilization device dry. Any risk of a water droplet being formed on the outer surface of the sterilization device due to condensation must be eliminated, as such droplet could potentially drop into the packaging material that is to be sterilized. Thus, it is very important to control the ambient conditions keeping in mind the temperature of the sterilization device, in particular of its outer surface and vice versa. Advantageously, the heat exchange unit is thus adapted to provide the heat exchange between the at least one medium flow and the at least one gas flow.

According to one or more embodiments the at least one electron beam emitter comprises a first body and a second body, wherein the second body is adapted for insertion into a packaging container. Cross sections of the two bodies are expediently round, in particular circular, wherein a diameter of the first body is bigger than a diameter of the second body. According to one or more embodiments the first body comprises the cathode housing and the filament. The second body comprises the electron exit window. Expediently, the second body has a longitudinal form which allows an insertion e.g. into a packaging container, such as a carton or PET packaging container. The diameter of the first body is preferably bigger which minimizes the risk of creating electrical arcs inside the housing. The above mentioned vacuum chamber is formed by the second body and at least partly by the first body. According to one or more embodiments the first body is adapted to be connected to the power supply unit e.g. via a high voltage output connector of the at least one power supply unit. In general, a plurality of sterilization devices is arranged at a movable or rotatable carousel or carrier plate.

As mentioned before, the at least one gas flow can be dried. Thus, according to one or more embodiments, the temperature of the at least one gas flow is decreased or decreasable due to the heat exchange. In other words, the at least one medium flow is used to cool the at least one gas flow. This means that according to one aspect of the invention an inlet temperature of the at least one medium flow is lower than an inlet temperature of the at least one gas flow into the heat exchange unit. There are no limitations concerning a design of the heat exchange unit. In addition, also more than one heat exchange unit can be used. According to one or more embodiments, the heat exchange unit is for example a parallel flow heat exchanger or a counter flow heat exchanger. According to another aspect of the invention the heat exchange unit comprises e.g. pipes in which the (cooling) medium is provided. In other words, the pipes guide the medium flow or the at least one medium flow. The relatively warmer gas flow condensates on the outside of the pipes and when contacting the pipes, the gas flow is cooled down. At the same time, the (cooling) medium flow inside the pipes heats or gets warmer. An outlet temperature of the at least one gas flow can be basically equal to an outlet temperature of the at least one medium flow. A temperature difference between these two temperatures depends on a performance of the heat exchange unit. As a consequence, the at least one gas flow and the at least one medium flow can have more or less the same temperature when leaving the heat exchange unit.

This leads to a dew point of the at least one gas flow that is very close to the temperature of the outlet temperature of the at least one medium flow (out of the heat exchange unit).

According to one or more embodiments, a flow direction of the at least one medium flow is directed from the heat exchange unit to the at least one device. In particular, the at least one medium flow is used to cool the at least one sterilization device or its power supply unit, respectively. According to one aspect of the invention the power supply unit comprises an electric system that is adapted to generate and provide the high voltage that is needed to operate the electron beam emitter. Expediently, the electric system of the power supply unit comprises power electronic components, high voltage components and control system components. One of the high voltage components is for example a voltage multiplier that is adapted to multiply an input voltage up to the high voltage that is needed to operate the electron beam emitter. According to one or more embodiments the medium flow is adapted to keep a temperature level of the electric system within a range of about 15 to 25° C. In general, 20° C. is a preferred value for the components of the electric system. It goes without saying that the temperature of the medium flow increases while cooling the power supply unit or its components, respectively. Thus, as the temperature level of the gas flow and the medium flow were basically the same leaving the heat exchange unit, the temperature of the medium flow is now, in any case, higher than the temperature of the medium flow. Nevertheless, advantageously the (already heated) medium flow can still be used for cooling.

According to another aspect of the invention the flow direction of the at least one medium flow is directed from the power supply unit to the at least one electron beam emitter, wherein the at least one medium flow is preferably used to cool the electron exit window of the at least one electron beam emitter. Generally, the temperature level of the power supply unit is lower than a temperature level of the at least one electron beam emitter. This means that the at least one medium flow that has already heated up during cooling of the power supply unit can still be used for cooling the electron beam emitter and in particular of its electron exit window. According to one or more embodiments the electron exit temperature is cooled by the medium flow so that a temperature of the electron exit window lies within a range of about 200° C. Relating to the idea of the invention, it is a big advantage that the electron exit window is cooled with a medium flow that is in any case warmer than an ambient temperature of the electron exit window. Having passed the heat exchange unit, the at least one medium flow and the at least one gas flow have basically the same temperature levels. Having passed the power supply unit, the temperature of the medium flow is in any case higher than the temperature of the gas flow (having passed the heat exchange unit, where it is heated up a bit). In a last step, the already heated medium flow is used to cool the electron beam emitter and in particular its electron exit window. There is no risk that the temperature of the electron exit window falls below a temperature of the ambient air, as the electron exit window is cooled with the medium flow that has a temperature that is higher than the temperature of the ambient air. No condensation can occur on the cooled surfaces of the emitter or in particular at the electron exit window as the dew point of the ambient air is lower than the temperature of the electron exit window.

In this context it has to be mentioned that condensation is especially a problem when the sterilizations process is stopped or generally during shutdown of the sterilization device or the sterilization machine, respectively. During this time, there is the risk that for example a temperature of the electron exit window falls below an ambient temperature. Condensation could occur. However, the at least one medium flow is also adapted to heat the at least one device, such as the electron exit window. Thus, it can be ensured that the temperature of the device or a temperature of the outer surface of the device, respectively, is always higher than an ambient temperature of the device, hence higher than the dew point so no condensation occurs.

Generally, according to one or more embodiments an ambient temperature of the at least one device is lower than a surface temperature of the at least one device. The aforementioned features and advantages do not explicitly refer only to the electron exit window. They are rather valid for the whole device, in particular for the sterilizations device, and its outer surface as a whole, especially, those parts which interact with the part to be sterilized such as the package.

According to one or more embodiments a flow direction of the at least one gas flow is directed from the heat exchange unit to the at least one device. In general, the at least one gas flow is adapted to condition, control or adjust, respectively, the ambient conditions of the sterilization device or of a plurality of sterilization devices. Thus, it should be made sure that the ambient temperature of the sterilization device or of the sterilization devices corresponds to the temperature of the at least one gas flow. According to one or more embodiments a plurality of gas flows is provided that are directed to the sterilization device(s). In addition, also the use of one or more means for conveying, as for example fans, can be provided to adjust and/or control the ambient conditions. Relating to the sterilization devices, expediently also a plurality of medium flows is provided that are all directed from the heat exchange unit to the appropriate sterilizations devices. Possibly, a medium flow can also be guided from one sterilization device to the next, in particular if the medium flow can be cooled down in between, e.g. by an appropriate heat exchanger.

As already mentioned, according to one aspect of the invention an inlet temperature of the at least one medium flow into the heat exchange unit is lower than an inlet temperature of the at least one gas flow into the heat exchange unit. Since the medium flow heats during cooling the device, in particular the sterilization device, is has to be ensured that the inlet temperature of the medium flow into the heat exchange unit is lower than the inlet temperature of the gas flow into the heat exchange unit.

According to one or more embodiments, the at least one medium flow is a circulating medium flow. This means that according to one aspect of the invention the at least one medium flow is directed from the at least one device to the heat exchange unit. In this case, as mentioned before, it has to be used e. g. a heat exchanger to make sure that the inlet temperature of the medium flow into the heat exchange unit is lower than the inlet temperature of the gas flow into the heat exchange unit.

According to one or more embodiments the conditioning system comprises a housing, wherein the housing is adapted to encase the at least one device. According to an aspect of the invention, the housing comprises appropriate inlets and outlets for the at least one gas flow. Such housing can help to control and adjust the ambient conditions of the (sterilization) device(s), i.e. the temperature of the ambient gas. A housing may also comprise a plurality of sterilization devices. As the sterilization devices are generally arranged at a movable carousel or carrier plate, the housing or a plurality of housings (for each sterilization device) can protect the ambient area of the sterilization device or of the sterilization devices e.g. from upcoming air flows etc.

According to the invention, a sterilization machine, in particular for packaging material, comprises a plurality of sterilization devices and at least one conditioning system, wherein the conditioning system comprises a cooling system, at least one gas flow and a heat exchange unit, wherein the at least one gas flow is adapted to adjust ambient conditions of the plurality of sterilization devices, wherein the cooling system comprises at least one medium flow, and wherein the at least one medium flow is adapted to cool and/or heat the plurality of sterilization devices, characterized in that the heat exchange unit is adapted to provide a heat exchange between the at least one medium flow and the at least one gas flow.

According to the invention, a method to condition ambient conditions of a device, in particular a sterilization device, comprises the steps:
providing a gas flow that is adapted to adjust ambient conditions of at least one device, in particular a sterilization device;
providing at least one medium flow that is adapted to cool the at least one device;
providing a heat exchange between the at least one medium flow and the at least one gas flow.

The conditioning system according to the invention can include the features and advantages of the sterilization machine according to the invention and of the method to condition ambient conditions of a device according to the invention and vice versa.

Additional aspects and features of the current invention are shown in the following description of preferred embodiments of the current invention with reference to the attached drawings. Single features or characteristics of respective embodiments are explicitly allowed to be combined within the scope of the current invention.

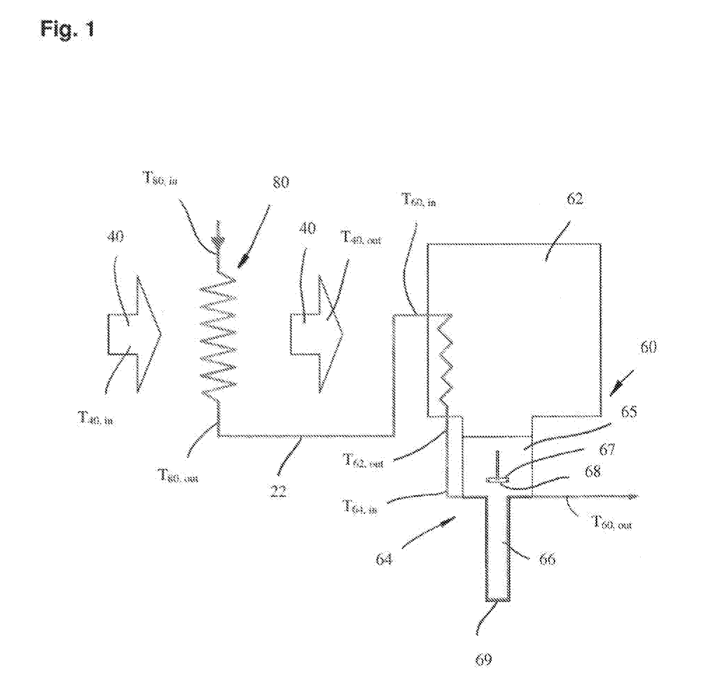

FIG. 1: shows a schematic diagram of a conditioning system;

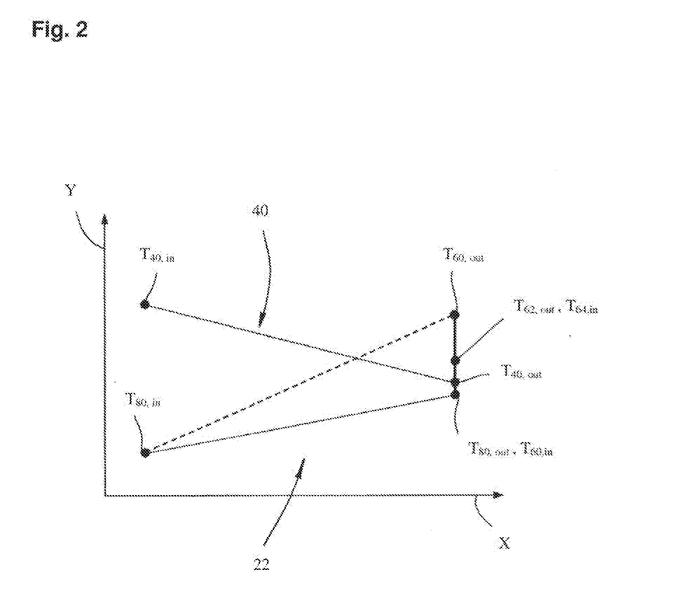

FIG. 2: shows schematic temperature profiles of at least one gas flow and at least one medium flow.

Referring now to FIG. 1 a schematic diagram of a conditioning system is shown. The conditioning system comprises a medium flow 22 that is directed from a heat exchange unit 80 to a sterilization device 60. A gas flow 40 is directed from the heat exchange unit 80 to the sterilization device 60. The gas flow 40 has an inlet temperature T_{40,in} and an outlet temperature T_{40,out.} The medium flow 22 has with reference to the heat exchange unit 80 an inlet temperature T_{80,in} and an outlet temperature T_{80,out}. With reference to the sterilization device 60, the medium flow 22 has an inlet temperature T_{60,in} and an outlet temperature T_{60,out.} As shown in FIG. 1, the medium flow 22 is guided from a power supply unit 62 to an electron beam emitter 64. Thus, according to this embodiment the medium flow 22 has also an outlet temperature T_{62,out} and an inlet temperature T_{64,in}. The electron beam emitter 64 comprises a first body 65 and a second body 66. The first body 65 comprises a cathode housing 67 and a filament 68. The elongate second body 66 of the electron beam emitter 64 comprises an electron exit window 69.

FIG. 2 shows schematic temperature profiles of a gas flow 40 and a medium flow 22 of a conditioning system as for example shown in FIG. 1. Reference numeral x indicates a flow direction in a heat exchange unit. Reference numeral y indicates a temperature axis. It is shown that a temperature of the gas flow 40, passing the heat exchange unit, decreases, wherein a temperature of the medium flow 22 increases. This causes a drying of the gas flow 40. An inlet temperature T_{40,in} of the gas flow 40 falls down to an outlet temperature T_{40,out}. According to one or more embodiments the heat exchange unit comprises e.g. pipes in which the (cooling) medium is provided. In other words, the pipes guide the medium flow 22 or the at least one medium flow 22. The relatively warmer gas flow 40 condensates on the outside of the pipes and when contacting the pipes, the gas flow 40 is cooled down. At the same time, the (cooling) medium flow 22 inside the pipes heats or gets warmer. The outlet temperature T_{40,out} of the gas flow 40 is basically on the same level as an outlet temperature T_{80,out} of the medium flow 22. Having passed the heat exchange unit, an inlet temperature T_{80,in} of the medium flow 22 has increased to an outlet temperature T_{80,out}. In other words, the temperature T_{40,out} of the gas flow 40 is equal or very close to the temperature T_{80,out} of the medium flow 22. A temperature difference between these two temperatures depends on a performance of the heat exchange unit. In general, having passed the heat exchange unit, a dew point of the at least one gas flow 40 is very close to the temperature T_{80,out} of the at least one medium flow 22.

Having passed the heat exchange unit, the at least one medium flow 22 is directed to the interior of the sterilization device, whereas the gas flow 40, having the temperature T_{40,out}, is directed inside the housing, i.e. to an ambient gas surrounding the sterilization device, in particular surrounding an electron exit window of the sterilization device.

According to one or more embodiments the at least one medium flow 22 is at first directed to the power supply unit, in order to cool the electric system that is located within the power supply unit or within its housing, respectively. Having passed the power supply unit, the temperature T_{62,out} of the medium flow 22 is in any case higher than the temperature T_{40,out} of the gas flow 40 (having passed the heat exchange unit). In a last step, the already heated medium flow 22 is used to cool the electron beam emitter and in particular its electron exit window. An outlet temperature T_{62,out} of the at least one medium flow 22 out of the power supply unit will be basically the same as an inlet temperature T_{64,in} of the medium flow 22 into the electron beam emitter. There is no risk that a temperature of e.g. an electron exit window falls below a temperature of the ambient air, as the electron exit window is cooled with the medium flow 22 that has a temperature that is in any case higher than the temperature T_{40,out} of the ambient air or the gas flow 40, respectively. No condensation can occur on the cooled surfaces of the emitter or in particular at the electron exit window. After the cooling process of the electron beam emitter, the at least one medium flow 22 has a temperature T_{60,out}. The invention is particularly useful when the inlet temperature T_{80,in} of the at least one medium flow 22 into the heat exchange unit is lower than the inlet temperature T_{40,in} of the at least one gas flow 40 into the heat exchange unit. The at least one medium flow 22 can be a part of open cooling circuit. However, also a closed cooling circuit or closed cooling circuits, respectively, can be realized, e.g. using appropriate heat exchanger. Finally, it has to be mentioned that the temperature levels shown in FIG. 2 are only used by way of example. They cannot represent certain quantitative temperature levels. However, they show in terms of quality the levels of the different temperatures relatively to each other.

The invention can be applied in for example an irradiation device as described in the international application No. PCT/EP2013/076870 filed by the applicant.

### REFERENCE NUMERALS

22 medium flow
40 gas flow
sterilization device
outer surface
power supply unit
electron beam emitter
65 first body
second body
cathode housing
filament
electron exit window
80 heat exchange unit, heat exchanger unit, air dryer
T_{80,in} inlet temperature of medium flow (heat exchange unit)
T_{80,out} outlet temperature of medium flow (heat exchange unit)
T_{60,in} inlet temperature of medium flow (sterilization device)
T_{60,out} outlet temperature of medium flow (sterilization device)
T_{62,out} outlet temperature of medium flow (power supply unit)
T_{64,in} inlet temperature of medium flow (electron beam emitter)
T_{40,in} inlet temperature of gas flow
T_{40,out} out temperature of gas flow
x flow direction (heat exchange unit)
y temperature axis

1. Conditioning system for at least one sterilization device, said sterilization device comprising at least one electron beam emitter, said conditioning system comprising: a cooling system, at least one gas flow and a heat exchange unit,
the at least one gas flow being configured to adjust a temperature of ambient gas around the sterilization device,
the cooling system comprising at least one medium flow, and
the at least one medium flow being configured to cool and/or heat the sterilization device, and
the heat exchange unit being configured to provide a heat exchange between the at least one medium flow and the at least one gas flow to avoid risk of condensation of the ambient gas on the sterilization device.

2. Conditioning system according to claim 1, wherein the sterilization device comprises a power supply unit.

3. Conditioning system according to claim 1, wherein a temperature of the at least one gas flow is decreased due to the heat exchange.

4. Conditioning system according to claim 1, wherein a flow direction of the at least one medium flow is directed from the heat exchange unit to the sterilization device.

5. Conditioning system according to claim 4, wherein the flow direction of the at least one medium flow is directed first to the power supply unit, wherein the at least one medium flow is adapted to cool the power supply unit, and then to the at least one electron beam emitter, wherein the at least one medium flow is adapted to cool an electron exit window of the at least one electron beam emitter.

6. Conditioning system according to claim 1, wherein the temperature of the ambient gas surrounding the sterilization device is lower than a surface temperature of the sterilization device.

7. Conditioning system according to claim 1, wherein a flow direction of the at least one gas flow is directed from the heat exchange unit to the at least one sterilization device.

8. Conditioning system according to claim 1, wherein an inlet temperature of the at least one medium flow into the heat exchange unit is lower than an inlet temperature of the at least one gas flow into the heat exchange unit.

9. Conditioning system according to claim 1, wherein the at least one medium flow is a circulating medium flow.

10. Conditioning system according to claim 1, comprising a housing, wherein the housing is adapted to encase the at least one sterilization device.

11. Sterilization machine, for sterilization of packaging material, comprising: a plurality of sterilization devices and at least one conditioning system,
the conditioning system comprising a cooling system, at least one gas flow and a heat exchange unit,
the at least one gas flow being configured to adjust the temperature of ambient gas around the plurality of sterilization devices,
the cooling system comprising at least one medium flow, and
the at least one medium flow being configured to cool and/or heat the plurality of sterilization devices, and
the heat exchange unit being configured to provide a heat exchange between the at least one medium flow and the at least one gas flow to avoid risk of condensation of the ambient gas on the sterilization devices.

12. Method of conditioning a sterilization device, comprising:
adjusting the temperature of ambient gas around the sterilization device by way of a gas flow;
colling and/or heating the at least one device by way of a medium flow;
providing heat exchange between the at least one medium flow and the at least one gas flow to avoid risk of condensation of the ambient gas on the sterilization device.
